(19) United States
(12) Patent Application Publication
(10) Pub. No.: US 2006/0224106 A1
(43) Pub. Date: Oct. 5, 2006

(54) Dual syringe with sieve

(76) Inventor: Tammy J. Honchel, Lexington, KY (US)

Correspondence Address: Tammy Honchel, 916 Springwood Court, Lexington, KY, 40515, US

(21) Appl. No.: 11/080,139

(22) Filed: Mar. 15, 2005

(51) Int. Cl. A61M 5/178

(52) U.S. Cl. 604/37

(57) ABSTRACT

A dual syringe with sieve that can safely administer water, feedings, and medication without clogging a feeding tube and also check residual tube feed and feeding tube placement with only one step attachment to the feeding tube is disclosed.

### FIELD OF THE INVENTION

This invention relates to a device for administering medications and/or nutritional substances via a feeding tube. More specifically, this invention relates to a dual syringe consisting of a piston syringe and/or bulb syringe combination with sieve, to help prevent clogging of feeding tubes and repetitive attaching and un-attaching to the feeding tubes.

### BACKGROUND OF THE INVENTION

Within the healthcare industry, it is always desired to have devices that can help eliminate the need for costly procedures and to decrease the need for repeated use of expensive devices. It is also important to prevent healthcare workers from being exposed to the patient's bodily fluids.

Feeding tubes can require different types of syringes that have to be attached and un-attached several times thereto. It also happens frequently that medications that are difficult to crush and dissolve clog the tubes causing the patient to endure replacement of another feeding tube. The multiple times of attaching and un-attaching of the syringes to the feeding tube is a concern due to diseases that can be transmitted via bodily fluids (i.e. stomach contents) that can possibly infect health care workers that perform these duties.

Consequently, a syringe that can perform both as a piston syringe for checking residual tube feed and placement verification of feeding tube and a bulb syringe with a sieve for administering water, medications and feedings while preventing clogging of the feeding tube is desired.

### BRIEF SUMMARY OF THE INVENTION

This invention has as its primary objective to provide a syringe having a sieve and both bulb and piston capabilities for use with a feeding tube in such a way that it greatly increases safety of operation and overall utility for both the patent and healthcare professionals. These and other objects and advantages of the invention will become readily apparent as the following description is read in conjunction with the accompanying drawings.

### BRIEF DESCRIPTION OF THE DRAWINGS

FIG. 1 is a perspective view of an exemplary embodiment of the present invention;

FIG. 2 shows the present invention with a sieve in a ready to use position;

FIG. 3 shows an overhead view of FIG. 2; and

FIG. 4 shows an alternative embodiment in which a sieve is located internally attached to the present invention.

### DETAILED DESCRIPTION OF THE PREFERRED EMBODIMENTS

Before explaining the disclosed embodiment of the present invention in detail it is to be understood that the invention is not limited in its application to the details of the particular arrangement shown, since the invention is capable of other embodiments. Also, the terminology used herein is for the purpose of description and not of limitation.

In today's society, with people living longer and with more debilitating diseases, it is necessary to care for people whose nutrition and medications have to be administered via a feeding tube. Examples of these tubes include gastric tubes, Dobbhoff tubes, Salem tubes, or one of the other numerous tubes that are either surgically inserted into the stomach, or that is inserted into the stomach by way of the nasal or oral cavity. These tubes often become clogged from the medications that are administered therein, thus requiring a new tube to be inserted.

Also, there are numerous medications that do not come in liquid form, therefore requiring that a nurse crush pills and administer them via the feeding tube. These crushed granules often do not properly dissolve and clot together, thus causing clogging of the feeding tube.

When a feeding tube gets clogged it often requires replacement, which is not only expensive but painful for the patient. It happens often that a patient has to endure numerous attempts to pass a feeding through the nasal cavity to stomach due to the tubing getting caught and curling up in the throat or going into the lungs instead of the stomach. In addition to the expense and pain that they must endure, patients may also miss some of their scheduled medications while waiting for the tube to be replaced or for placement verification by an X-ray.

Prior to medication administration, feeding tube placement must be verified as well as residual checks performed. This is accomplished by administering a small amount of air into the stomach by way of the piston syringe and then withdrawing the piston which creates a suction that pulls out any residual tube feed or gastric content from the stomach. A piston syringe is desired for this process because it has a rubber tip that forms an air tight seal.

The feeding tube must be flushed with water before medication administration as well as after the administration because there are some medications that are incompatible with tube feeds. This as well as medication administration, water or feeding boluses, should be done with a bulb syringe so that the water, medication, and feeding can enter the stomach through the feeding tube via gravity or minimal air pressure which can be created by squeezing on the bulb portion of the bulb syringe.

Currently a nurse must attach and un-attach a syringe several times to perform the task of checking placement, residual checks, medication administration as well as flushing the feeding tube with water to help prevent clogging because two different syringe types are required to perform these task.

Due to the clogging of the feeding tubes by the granules of pills that do not crush or dissolve easily, a sieve would be useful to catch these granules. The caught granules could be further crushed or dissolved to prevent clogging of the feeding tube.

In performing their duties nurses are often measured by time and efficiency. Accordingly, a device that prevents small granules from passing through a syringe and causing clogging of the tube that could be easily used without the repetitive attaching and un-attaching steps is desired.

Patients with debilitating diseases are more than ever being taken care of at home. At home, if a feeding tube gets clogged, a visit to a hospital may be required for replacement of the feeding tube if unclogging measures are unsuccessful. If a dual syringe with a sieve or a single syringe with a sieve were available, it could possibly prevent the clogging of the tube by preventing medication granules that are too big to pass through the feeding tube from entering and clogging it, thus preventing unnecessary hospital visits and hospitalizations.

Due to the lack of time that nurses have, they often will use the piston syringe for both checking of residual tube feeding or gastric content and for administering meds, water boluses and bolus feedings. This is not wise because a nurse could apply too much pressure and force causing the feeding tube to rupture. This could also cause the patient to get sick because of the rapid administration of the substances. This is why a bulb syringe, rather than a piston syringe, is desired for the administration of bolus feedings, medications, and water flushes.

Meanwhile, a piston syringe is desired for checking of tube feed residual and placement of the feeding tube because the piston syringe has a rubber tip piston that creates an air tight seal, thus creating suction when pulled back.

Accomplishing all of these procedures would be much easier if a nurse had a dual syringe as described herein regarding the present invention. Using the present invention, all the activities described herein could be performed with the dual syringe and with only one connection to the feeding tube. However, if a dual syringe were undesirable, a single syringe with a sieve would still help prevent clogging.

The need to limit the times a syringe is attached and un-attached is not only important due to limited time, but also to decrease exposure of the health care worker (i.e. nurse) to possible deadly diseases that are contained in an infected patients bodily fluids, and that can be transmitted to a healthcare worker who may come in contact with these bodily fluids. It is often the case when attaching and un-attaching a syringe to check residuals, administer water, bolus feedings, and medications, that stomach content will flow back out of the tube and onto the health care workers hands. There are also times when a patient may cough or have air trapped in the stomach that may cause projectile emission of stomach contents that could expose a health care worker to infected bodily fluids. For all these reasons, it is desired to avoid repeatedly attaching and un-attaching a syringe to the feeding tube if possible.

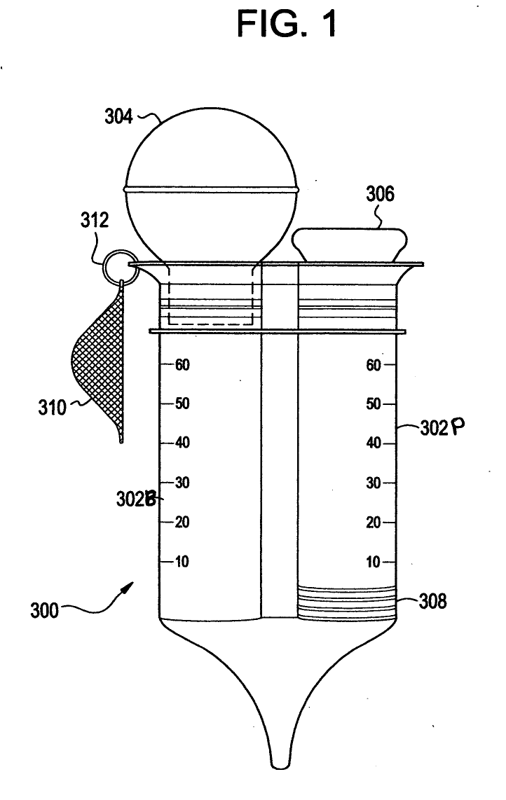

FIG. 1 depicts an exemplary embodiment of the dual syringe and sieve device. From FIG. 1 it is apparent that the dual syringe 300 consists of two separate syringe devices, a piston 306 and a bulb 304. The bulb 304 is used to push liquids through the bulb side 302B of the syringe 300, while the piston 306 is used for pushing materials through the piston side 302P of the dual syringe 300. The rubber tip of the piston 306 is depicted by 308. The rubber tip 308 helps to create a seal which helps to create suction when pulling back on the piston. This is important when checking for residual tube feed or gastric content in the stomach.

FIG. 1 also shows a sieve 310 attached to the syringe 300 by a ring 312, although other attachment means could also be employed so that the present invention should not be considered as limited exclusively thereto. The syringe 300 is carefully constructed to never intermix the contents of 302B with 302P, or vice versa.

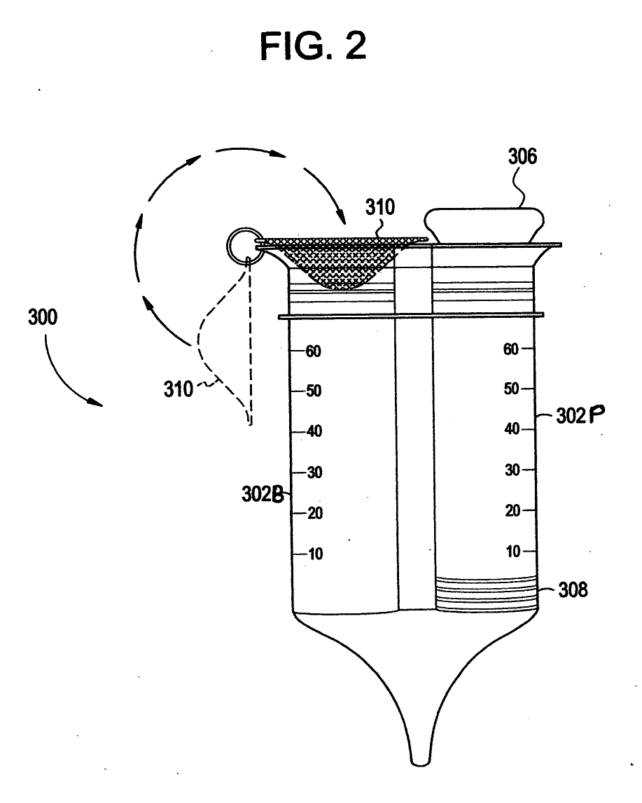

FIG. 2 shows a view of the dual syringe 300 with the sieve 310 in use and with the bulb portion 304 removed. The series of clockwise arrows in FIG. 2 depict how the sieve 310 can be raised and inserted into the upper opening of the bulb syringe portion 302B of the dual syringe 300. When the sieve 310 is in place, no undiluted or incompletely or improperly crushed substances can flow into the syringe 300.

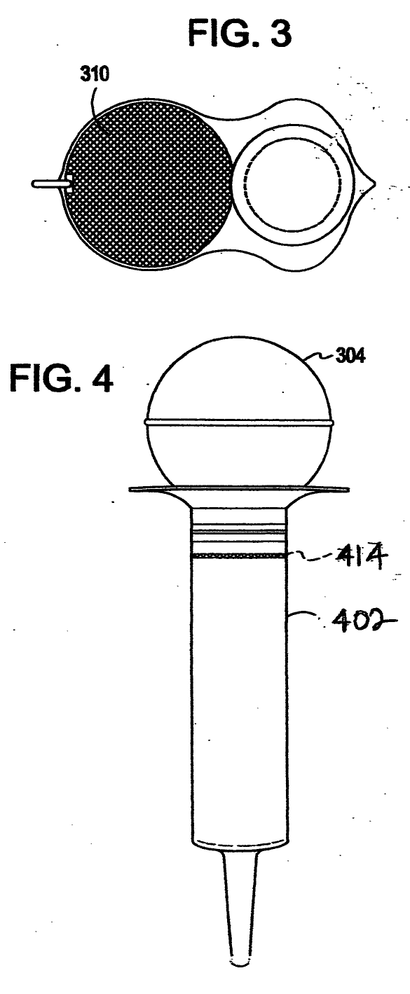

FIG. 3 shows an overhead view of the dual syringe 300 with the sieve 310 in place.

FIG. 4 shows an alternative single non-dual embodiment of the present invention, in which a single bulb syringe 402 has a sieve 414 that is manufactured to be permanently located therein. Thus, the present invention can have a sieve located either external or internal to either single syringe 402 or dual syringe 302.

It is anticipated that various changes may be made in the arrangement and operation of the system of the present invention without departing from the spirit and scope of the invention, as described in the following claims.

What is claimed is:

1. A dual-chamber syringe, comprising:
a tubular bulb chamber connected together in parallel with a tubular piston chamber so as to form a single injecting mechanism, wherein said bulb and piston chambers are segregated so that any fluids contained therein are prevented from intermingling,
said bulb section has an external sieve attached that can be placed in either of the syringe openings to filter any un-liquefied materials from passing therethrough.

2. The syringe of claim 1, wherein said sieve can also be permanently attached inside one or both of said chambers.

3. The syringe of claim 1, further comprising:
a second tubular portion with a rubber tipped plunger device used to check placement by administering a small amount of air and to withdraw stomach content as well as any residual tube feeds.

4. The syringe of claim 1, wherein said tubular portions of said device is composed of a plastic material.

5. The syringe of claim 1, wherein said bulb and tip of plunger portion are made of a rubber material.

6. The syringe of claim 1, wherein said sieve that is either external or internal to one or both of said chambers.

7. The syringe of claim 1, wherein said sieve is made of metal or plastic.

8. The syringe of claim 1, wherein said device is a disposable single patient use item.

9. The syringe of claim 1, wherein said device will allow healthcare workers to perform both checking of residuals, placement of the feeding tube, as well as administration of liquefied substances into the stomach via the feeding tube with one device.

10. A syringe, comprising:
a tubular bulb chamber with an injecting tip;
an external sieve that can be either detached or engaged so that when engaged, said sieve acts to filter any un-liquefied materials from passing through said syringe.

11. A method of utilizing a feeding tube connected to a medical patient, compromising:
checking for proper placement of said feeding tube;
pulling up a small amount of air into said piston syringe portion of said dual syringe;
attaching said dual syringe to said feeding tube; and
pushing on said piston to administer air into said stomach while listening through a stethoscope for bubbling in said stomach.

12. The method of claim 11, further compromising;
checking for residual tube feed and/or gastric content; pulling back on said piston portion of said piston syringe creating a mini-vacuum suction, withdrawing any stomach content e.g. residual tube feed, measuring the residual, and then push on said piston to reinsert said residual back into the stomach.

13. The method of claim 11, further compromising;
removing said bulb from said bulb syringe;
pouring a substance into said bulb syringe; thereby
allowing substance to enter the stomach through said feeding tube by gravity.

14. The method of claim 13, further compromising;
reapplying said bulb; and
squeezing said bulb; thereby
applying slight air pressure behind said substance; thereby
pushing the substance into the feeding tube and into the stomach.

15. The method of claim 11, further compromising;
removing said bulb from said bulb syringe;
applying said sieve;
pouring said medications through said sieve into said bulb syringe portion of said dual syringe; thereby
further dissolving any medications that are un-liquefied so they may pass through said sieve and said feeding tube without causing clogging of said feeding tube.

16. The method of claim 15, wherein said bulb and said piston are contained within the same syringe and all steps can be performed with a one step attachment and un-attachment of said dual syringe to said feeding tube.

17. The method of claim 11, further compromising;
removing said bulb from said bulb syringe;
applying said sieve;
pouring said medications through said sieve into said bulb syringe portion of said dual syringe; thereby
allowing medication to flow through said bulb syringe into the stomach through said feeding tube via gravity or by removing said sieve and replacing said bulb and applying slight pressure to said bulb to apply air pressure behind said medications thus pushing medications into the stomach.

18. The method of claim 17, wherein said bulb and said piston are contained within the same syringe and all steps can be performed with a one step attachment and un-attachment of said dual syringe to said feeding tube.
